(19) United States
(12) Patent Application Publication
(10) Pub. No.: US 2021/0146105 A1
(43) Pub. Date: May 20, 2021

(54) MICRONEEDLE AND METHOD FOR MANUFACTURING MICRONEEDLE

(71) Applicant: FEROKA Inc., Seoul (KR)

(72) Inventors: Induk Lee, Seoul (KR); Yeo Myung Lim, Seoul (KR); Yiseul Jeon, Seoul (KR); Jeongsun Nam, Seoul (KR)

(21) Appl. No.: 17/046,354

(22) PCT Filed: Mar. 15, 2019
(86) PCT No.: PCT/KR2019/003043

§ 371 Date: Oct. 9, 2020

(30) Foreign Application Priority Data
Apr. 10, 2018 (KR) 10-2018-0041536
Jan. 21, 2019 (KR) 10-2019-0007301

(51) Int. Cl. A61M 37/00; B29C 64/118; B33Y 10/00; B33Y 80/00; B29C 64/209; B33Y 30/00

(52) U.S. Cl. CPC A61M 37/0015; B29C 64/118; B33Y 10/00; B33Y 80/00; B29L 2031/7544; B33Y 30/00; A61M 2037/0053; A61M 2037/0023; A61M 2037/0046; B29C 64/209

(57) ABSTRACT

Provided are a microneedle using a laminated manner, a method of manufacturing a microneedle, and a system thereof. The method of manufacturing the microneedle according to an embodiment of the present invention includes extruding a first material using a first nozzle and extruding a second material using a second nozzle, and manufacturing the microneedle through a laminated manner using the extruded first material and second material.

### CROSS REFERENCE TO RELATED APPLICATIONS

This application is a National Stage application of International Patent Application No. PCT/KR2019/003043, filed on Mar. 15, 2019, which claims priority to Korean Patent Application No. 10-2018-0041536 filed on Apr. 10, 2018 and Korean Patent Application No. 10-2019-0007301 filed on Jan. 21, 2019, each of which is hereby incorporated by reference in its entirety.

### TECHNICAL FIELD

The present invention relates to a technology of manufacturing a microneedle, and more particularly, to a microneedle, which increases number density, improves an aspect ratio, and is capable of being manufactured in a multi-layered structure by manufacturing the multi-layered microneedle using a laminated manner, a method of manufacturing a microneedle, and a system thereof.

### BACKGROUND

When a bioactive substance is injected into a human skin, the existing injection needle may be used. Here, pain at an injection site, bleeding from skin damage, and disease infection due to the injection needle may be caused.

In recent years, a method of delivering a bioactive substance into the skin using a microneedle (or, an ultrafine needle) has been actively studied. The microneedle may have a diameter of tens to hundreds of micrometers to penetrate a stratum corneum of the skin, a main barrier layer.

The microneedle, unlike the conventional injection needle, may be characterized by painless skin penetration and no external damage. In addition, some degree of physical hardness may be required for the microneedle to penetrate the stratum corneum of the skin. In addition, an appropriate length may be required for the bioactive substance to reach an epidermis or a dermis of the skin.

In addition, in order to effectively deliver the bioactive substance of hundreds of the microneedles into the skin, the microneedle should have high skin permeability and be maintained for a certain period until dissolution after being inserted into the skin.

The conventional methods of manufacturing the microneedle include a mold manufacturing method and a tensile manufacturing method.

In the method of manufacturing the microneedle using the mold manner, it is difficult to penetrate the skin because an aspect ratio of the microneedle is low due to characteristics of the mold and the number density of the microneedle is low.

The method of manufacturing the microneedle using the tensile manner is a method of manufacturing by dropping a material on a patch, stretching the patch on which the material is dropped, drying the patch on which the material is dropped, and cutting out a thinned part. Due to the characteristics, there are problems in that a length of the microneedle is not constant and a lot of pain is felt due to a shape created.

In addition, both the mold manner and the tensile manner are very expensive to act as an obstacle to market growth and there is an inconvenience that the microneedle should be attached for about 2 hours because the microneedles are not densely arranged.

In addition, because it is difficult to increase the number density of microneedle in both methods, it is recommended to attach a microneedle patch manufactured by both methods for at least 2 hours, which takes too much time compared to a general pack that recommends attaching about 20 minutes. The reason why the attachment time is long is that the number density of needles is low. The number density of needles is low to cause the overall surface area of the needles included in the patch to be narrow and to cause a contact area with the skin to be narrow, thereby causing reaction speed with the skin to be inevitably slow. However, it is difficult to further increase the number density with the two existing methods, and therefore the reaction speed with the skin cannot be accelerated.

When considering production of a medical microneedle, not cosmetic purposes, limitation of the two conventional methods is further revealed. When vaccines or drugs are mixed in both conventional methods, the vaccines or drugs should be made into a homogeneous mixture of the same concentration in every needle. However, it is difficult to make the needle size constant and degree of penetration into the skin is not capable of being controlled due to the drugs remaining at an interface between the patch and the skin or in an injection passage, and thus administration in dose is close to impossible.

Accordingly, a need for a microneedle having a multi-layered structure has begun to be raised, and for example, in a case of insulin administration in dose, there has been suggested that the multilayered microneedle is required (Ito et al., Diabetes Technology & Therapeutics, 2012, October, 14).

Therefore, a method of manufacturing the multi-layered microneedle using a laminated manner which improves the number density of the microneedle, increases the aspect ratio, and allows various kinds of vaccine mixtures or drug mixtures to be arranged to be customized and to be administered in required dose is suggested.

### SUMMARY

### Technical Problem

Embodiments of the present invention provide a microneedle, which increases number density, improves an aspect ratio, and is capable of being manufactured in a multi-layered structure by manufacturing the multi-layered microneedle using a laminated manner, a method of manufacturing a microneedle, and a system thereof.

Embodiments of the present invention suggest a microneedle strengthening preservation of drugs and facilitating penetration into a skin by manufacturing a tree-shaped microneedle with three or more layers which includes a middle portion including a cavity having drugs, a lower portion supporting the middle portion, and an upper portion disposed on the middle portion, to facilitate penetration of the microneedle, a method of manufacturing the microneedle, and a system thereof.

### Technical Solution

A method of manufacturing a microneedle according to an embodiment of the present invention includes extruding a first material using a first nozzle and extruding a second material using a second nozzle, and manufacturing the microneedle through a laminated manner using the extruded first material and second material.

The manufacturing of the microneedle may include manufacturing the microneedle through a 3D printing manner using the first material and second material.

The extruding may include extruding the first material by a predetermined first extrusion sequence and extruding the second material by a predetermined second extrusion sequence.

The manufacturing of the microneedle may include manufacturing the microneedle through a laminated manner reflecting a mixing ratio of the first material and the second material.

A method of manufacturing a microneedle according to an embodiment of the present invention includes extruding a plurality of materials using at least two or more nozzles and manufacturing a microneedle through a laminated manner using the extruded plurality of materials.

A microneedle manufacturing system according to an embodiment of the present invention includes a first nozzle device for extruding a first material using a first nozzle, a second nozzle device for extruding a second material using a second nozzle, and a controller that manufactures the microneedle through a laminated manner using the extruded first material and second material.

The controller may manufacture the microneedle through a 3D printing manner using the first material and second material.

The first nozzle device may extrude the first material by a predetermined first extrusion sequence, and the second nozzle device may extrude the second material by a predetermined second extrusion sequence.

The controller may manufacture the microneedle through a laminated manner reflecting a mixing ratio of the first material and the second material.

A microneedle according to another embodiment of the present invention includes a middle portion that penetrates into a skin and be formed of a compound containing a drug component, a lower portion that supports the middle portion, and an upper portion disposed on an upper end of the middle portion to facilitate penetration.

The upper portion and middle portion may have a pyramidal or conical shape, and the lower portion may have a prismatic or cylindrical shape.

A bottom diameter of the middle portion may be greater than a bottom diameter of the upper portion or a bottom diameter of the lower portion, and the bottom diameter of the upper portion may be larger than the bottom diameter of the lower portion.

A height and a bottom diameter of the upper portion may be determined by a cross-sectional area of a fore-end of a truncated pyramid or truncated cone of the middle portion.

The lower portion may be located in the lowermost layer of the three or more layers structure to be in a shape coupled to a bottom diameter of a pyramid or cone of the middle portion, and may be formed with a diameter capable of supporting the middle portion formed of a compound containing a drug component in company with the upper portion.

The lower portion may be formed of a melting material connecting a base portion to the microneedle to separate the microneedle from the base portion.

The middle portion may be formed of a compound containing a drug component and is solidified.

The upper portion, the middle portion, and the lower portion may be formed of different materials.

The microneedle may be manufactured through a 3D printing manner.

### Advantageous Effects of the Invention

According to embodiments of the present invention, the microneedle may be manufactured using the laminated manner to increase the number density and improve the aspect ratio.

In detail, according to embodiments of the present invention, after forming the plurality of materials, for example, the base, the vaccine, and the drug mixture using the laminated manner, the microneedle may be manufactured using the same, to increase the number density of microneedle, improve the aspect ratio, enable administration in dose, and adjust the dissolution sequence and speed of the drugs.

In addition, according to embodiments of the present invention, the microneedle may be manufactured using the laminated manner, for example, 3D printing technology, to have advantages in terms of technical and economic aspects, such as skin perforation, presence of pain, needle number density, attachment time, precision, price, and expandability, compared to the conventional manner.

In addition, the microneedle may be manufactured according to the present invention, to secure high competitiveness in the wrinkle-improving cosmetic market and the medical market.

That is, the present invention is suitable for medical use because it is possible to manufacture the multi-layered microneedle using the laminated manner.

### BRIEF DESCRIPTION OF THE DRAWINGS

FIG. 1 is a flowchart illustrating a method of manufacturing a microneedle using a laminated manner according to an embodiment of the present invention;

FIG. 2 illustrates an exemplary view for explaining a method according to the present invention;

FIG. 3 illustrates an exemplary view comparing a microneedle manufactured by a method according to the present invention and the conventional method;

FIG. 4 illustrates a configuration of a microneedle manufacturing system using a laminated manner according to an embodiment of the present invention;

FIG. 5 is a perspective view illustrating a microneedle according to another embodiment of the present invention;

FIG. 6 is a cross-sectional view of a microneedle including a cavity according to another embodiment of the present invention;

FIG. 7 is a cross-sectional view of a microneedle of three or more layers structure according to another embodiment of the present invention; and

FIG. 8 is a perspective view illustrating a microneedle patch manufactured according to another embodiment of the present invention.

### DETAILED DESCRIPTION

Hereinafter, embodiments according to the present invention will be described in detail with reference to the accompanying drawings. However, the present invention is not restricted or limited by the embodiments. In addition, the same reference numerals shown in each drawing denote the same member.

A gist of embodiments of the present invention is that a plurality of materials, for example, a base, a vaccine, and a drug mixture are stacked using a laminated manner and a microneedle is manufactured using the stacked plurality of materials to increase number density of microneedle and to improve an aspect ratio.

Here, the present invention may extrude a first material and a second material using a first nozzle for extruding the first material, for example, the base material, and a second nozzle for extruding the second material, for example, the vaccine, the vaccine mixture, or the drug mixture and may manufacture the microneedle including the first material and the second material using a laminated manner.

The laminated manner in the present invention may include all kinds of manners in which the first material and the second material are capable of being formed in the laminated manner, and as an example, a 3D printing manner or a 3D printing technology may be included.

Here, the 3D printing technology or the 3D printing manner refers to a manner of stereoscopically forming an subject having a desired shape and form using a three-axis control system and refers to a technology mainly applied to a 3D printer.

Further, the present invention may manufacture the microneedle by adjusting movement of the first nozzle and the second nozzle up and down or left and right or manufacture the microneedle by adjusting movement of a bed (or base) in which the microneedle is manufactured up and down or left and right.

A resource or material of the microneedle used in the present invention may be a vaccine, a vaccine mixture, or a drug mixture, the material may be contained in a chamber, and the resource or material contained in the chamber may be extruded through the nozzles to manufacture the base or bed, a bottom where the microneedle is fixed. Here, the base or bed may move up and down or left and right along a conveyor belt or motor.

A gist of other embodiments of the present invention may strengthen preservation of the drug, facilitate penetration into a skin, and allow administration of the drug in a liquid state by manufacturing the microneedle with three or more layers including a middle portion formed of a compound containing a drug component, an upper portion that is positioned on an upper end of the middle portion to facilitate penetration into the skin, and a lower portion supporting the middle portion. Here, the microneedle according to an embodiment of the present invention is characterized in that the microneedle has a structure of three or more layers.

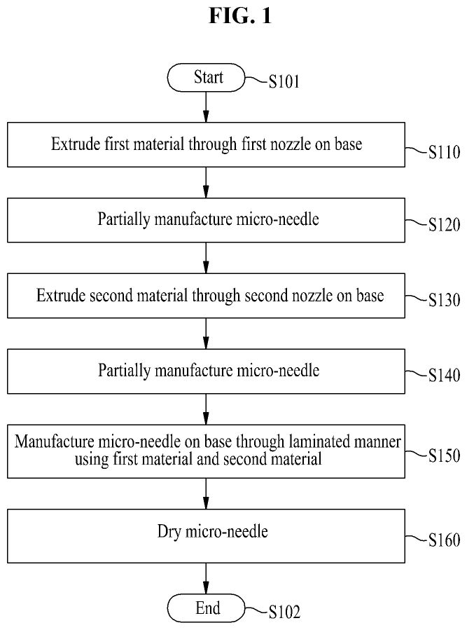

Hereinafter, an embodiment of the present invention will be described in detail with reference to FIGS. 1 to 8.

FIG. 1 is a flowchart illustrating a method of manufacturing a microneedle using a laminated manner according to an embodiment of the present invention, and as an example, illustrates a flowchart for a method of manufacturing a microneedle using a 3D printer.

Referring to FIG. 1, in a method of manufacturing a microneedle according to the present invention, a first material contained in a chamber is extruded to a base through a first nozzle, and the microneedle is partially manufactured on the base in S110 and S120.

Here, the first nozzle may be formed by extrusion mold of a perforated plate with a plurality of holes and the first material, for example, a base material of the microneedle, may be extruded on the base through the first nozzle.

When the microneedle using the first material may be partially manufactured by S110 and S120, a second material contained in anther chamber is extruded to the base through a second nozzle to partially manufacture the microneedle on the base again in S130 and S140.

Here, the second nozzle, like the first nozzle, may be formed by extrusion mold of a perforated plate with a plurality of holes and the second material may be extruded on the first material on the base to manufacture the microneedle in which the first material and the second material form a stacked structure.

In the present invention, a hole size of the first nozzle extruding the first material and a hole size of the second nozzle extruding the second material may be determined in consideration of materials to be extruded, an aspect ratio of the microneedle to be manufactured, and a mixing ratio of the first material and the second material.

Operations S110 to S140 may be repeatedly performed depending on the number of stacks to be laminated. In addition, operations S110 to S140, that is, a process of extruding the first material and the second material on the base may be performed using a laminated manner, for example, a 3D printing technology to manufacture a multi-layered microneedle including the first material and the second material and the microneedle as manufactured above dries in 150 and 160.

Here, operation S160 of drying the microneedle may be performed between a process of extruding the first material and a process of extruding the second material on the base in parallel, to perform drying with the extrusion of each material. The drying process may be determined by a person skilled in the art of manufacturing the microneedle.

In the method of the present invention, operations S110 and S130 may be repeatedly performed depending on a situation, and an extrusion time of the first material, an extrusion sequence of the first material, an extrusion time of second material, and an extrusion sequence of the second material may vary in consideration of the mixing ratio of the first material and the second material.

For example, in the present invention, after extruding the first material for a first time, the second material may be extruded on a top of the first material for a second time, and again, after extruding the first material for a third time on a top of the second material, the second material may be extruded on a top of the first material for a fourth time, thereby manufacturing the microneedle.

In the method according to the present invention, the stacked structure of the first material and the second material forming the microneedle, the extrusion time or extrusion sequence of the first material, the extrusion time or extrusion sequence of the second material may be determined in consideration of a hole size of each nozzle and a stacking height.

In addition, in the method according to the present invention, the base material and a vaccine material as well as the base material and a plurality of drugs or vaccines may be arranged in the microneedle. For example, when a microneedle containing two vaccines is manufactured, the base material, a first vaccine mixture, and a second vaccine mixture may be filled in each of three chambers and may be sequentially extruded through nozzles provided in each chamber or extruded through a predetermined order and extrusion sequence, thereby manufacturing the microneedle containing a plurality of vaccines. Of course, a ratio for the plurality of vaccines included in the microneedle may be predetermined and manufactured and the microneedle including the above-described ratio may be manufactured by adjusting the extrusion sequence and the extrusion time.

Furthermore, the method according to the present invention may use the laminated manner, thereby precisely adjusting an amount of vaccine contained in the microneedle. The microneedle manufactured in the above-described way may be made into a microneedle patch and may be easily applied to the medical field. That is, the present invention may manufacture the microneedle using the laminated manner to secure high competitiveness in a medical market field.

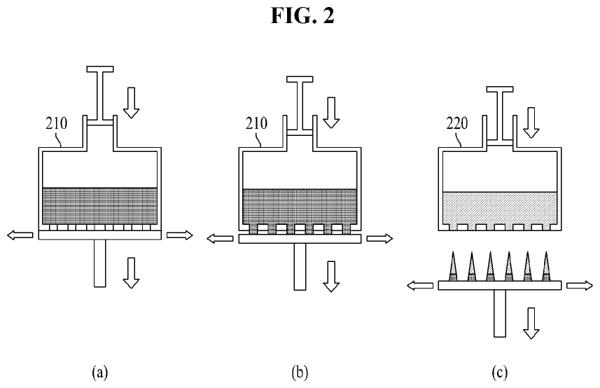

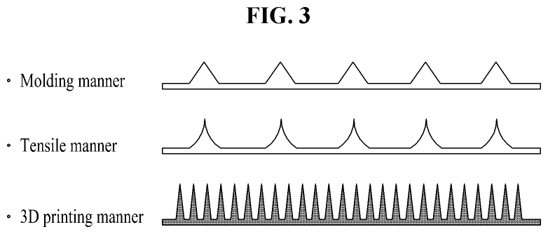

The invention will be described with reference to FIGS. 2 and 3 as follows.

FIG. 2 illustrates an exemplary view for explaining a method according to the present invention and illustrates an exemplary view for manufacturing a microneedle while a base or bed moves in three axes of x, y, and z.

As shown in FIGS. 2A and 2B, a first material contained in a first chamber 210 may be extruded in a specific extrusion sequence to extrude a first material on a base through a first nozzle. Of course, the extrusion sequence and movement of the base may be achieved through control of a system or apparatus for manufacturing the microneedle of the present invention.

As shown in FIG. 2C, when the first material is extruded on the base, a second material, for example, a vaccine mixture contained in a second chamber 220 is extruded in a specific extrusion sequence, thereby extruding the second material on the first material formed on the base through a second nozzle.

The multi-layered microneedle including the first material and the second material may be manufactured through the extrusion sequence and movement of the base.

Accordingly, the microneedle manufactured by the laminated manner may improve number density, increase an aspect ratio, enable quantitative administration, and control dissolution sequence and speed of the drug compared to the conventional mold manner and tensile manner. Of course, the number density and aspect ratio may be controlled by the method according to the present invention, and further, when a plurality of vaccines or drugs are to be included in the microneedle, the microneedle may be easily manufactured using the laminated manner.

Although, in FIG. 2, it is described that the base or bed moves in the three-axis of x, y, and z to manufacture the multi-layered microneedle, the present invention is not limited thereto, the chamber or nozzle may move in the three-axis of x, y, and z to manufacture the multi-layered microneedle, and the chamber or nozzle as well as the base or bed may move in the three-axis of x, y, and z to manufacture the multi-layered microneedle.

FIG. 3 shows an exemplary view comparing microneedles manufactured by a method according to the present invention and the conventional method.

Referring to FIG. 3, it may be seen that the mold manner and the tensile manner to make the microneedles have low number density, whereas the microneedle manufactured using the laminated manner, for example, the 3D printing manner, have very high number density compared to the conventional manner due to limitations of the mold manner and the tensile manner and the microneedle manufactured using the method of the present invention has higher aspect ratio than those of the microneedles manufactured using the mold manner and the tensile manner. Of course, the method according to the present invention may adjust the aspect ratio of each microneedle and the aspect ratio may be determined by fields in which the microneedle of the present invention is used, for example, for treatment, medical use, and the like.

Table 1 below is a comparison of the conventional mold manner and tensile manner, and the manner (3D printing) according to the present invention.

TABLE 1

| | Mold manner | Tensile manner | 3D printing |
|---|---|---|---|
| Skin perforation | X | ◯ | ◯ |
| Presence or | Absence | Presence | Absence |
| absence of pain | | | |
| Needle number | Low | Low | High |
| density | | | |
| Attachment time | 2 hours | 2 hours | Within 10 |
| | | | minutes |
| Precision | High | Low | High |
| Price | Expensive | More expensive | Cheaper than |
| | | than the mold | mold manner |
| | | manner | |
| Extensibility | None | Some | High |

As seen from Table 1, it may be seen that the method (3D printing) according to the present invention is advantageous in skin perforation, no pain, and has the higher number density of the microneedle compared to the mold manner and the tensile manner. In addition, it may be seen that the method (3D printing) according to the present invention has the much shorter attachment time and higher precision compared to the conventional manner, and the manufacturing cost is lower due to using the laminated manner, for example, the 3D printing manner, thereby having higher extensibility compared to the conventional manner. As described above, the method according to the present invention is a very advantageous in terms of technical and economical aspects compared to the conventional mold manner and tensile manner.

That is, according to the method of the present invention, the microneedle implemented by the lamination technology has the high aspect ratio, and therefore, the skin perforation is also good, pain is very low, and the attachment time is very short due to the high number density. In addition, the microneedle with a high precision of about 5 micrometers may be implemented and a desired drug mixture may be arranged in a desired position, and thus the extensibility is high.

Furthermore, in the present invention, a nozzle, which adjusts extrusion speed and has a hole of a desired size, may be replaced in consideration of characteristics of the resource or material, for example, viscosity, time taken for curing, and the like.

For example, in the present invention, when two or more chambers are used and there is a difference in speed in each operation, for example, when the moving speed of the base and the time required for each chamber during the injection processes of the materials are different, the extrusion speed for each chamber may be changed or the size of the nozzle hole may be changed. In addition, a process schedule may be adjusted to minimize waiting time between the chamber extrusion process and the next chamber extrusion process. For example, when there are an extrusion process “A” of the first chamber and an extrusion process “B” of the second chamber, and the process “B” proceeds after the process A, a system for the process “A” simultaneously processes the next product. Here, when the process “A” is shorter than the process “B”, the process “A” may start after waiting for a required time difference to be finished at the same time as the process “B”. Otherwise, when the process “B” is shorter than the process “A”, a system for the process “B” may start working as soon as a result is received from the system for the process “A”.

The curing method in the present invention may use a variety of methods, for example, the curing may be performed in a form in which air in a dry state is blown to the microneedle to be circulated and the microneedle may be cured using a desiccant together when it is necessary to maintain a clean room.

In addition, when the microneedle is manufactured using the method according to the present invention, various problems may occur and these problems may be solved in the following method.

1) When the nozzle hole is hardened and clogged between operations during the extrusion process of the material, a close cover is put on the nozzle to prevent air from coming in contact with the nozzle hole after the extrusion is finished, a hardened part of the nozzle may be removed by extrusion, and a bottom of the nozzle may be scraped each time before extrusion to keep a nozzle portion clean.

2) When a shift occurs in a device such as a motor for moving the chamber or the base, an encoder or image information is analyzed and corrected to remove possible shift.

3) When the material sticks to the bottom of the nozzle, the bottom may be scraped to be solved. In addition, when a problem occurs depending on material types, the bottom of the nozzle may be coated with a low-reactivity material such as Teflon or the nozzle may be shaped to protrude.

4) An efficient method of exchanging chambers when manufacturing the microneedle using two or more chambers may be optimized in consideration of both the movement of the nozzle and the movement of the base, or may arrange the chambers to provide a variety of movement paths. Of course, in the present invention, the chamber may be arranged in consideration of adding a chamber or manufacturing a microneedle having two or more layers.

5) When the base is moved using a conveyor belt, alignment between the base and the chamber may be rearranged through an algorithm that analyzes image information and allows the chamber to find its original position.

In addition, the present invention may check whether there is a defect for the manufactured microneedle and a method for determining whether there is the defect or not may include analyzing an image of the manufactured microneedle or automatically analyzing and checking a shape, arrangement, and layer structure of the microneedle though image analysis of the microneedle in each process. It may be possible to do total inspection automatically through the above-described process.

Further, a process of filling the chamber with the material used in the present invention may include receiving the material from a large container and the large container may maintain a sealed state through a piston or the like to prevent contamination of the material or may be injected with aseptic drying air.

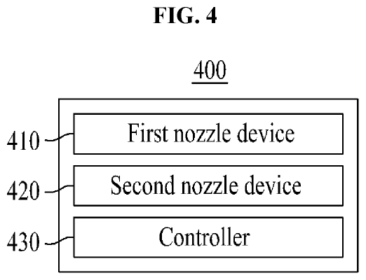

FIG. 4 illustrates a configuration of a microneedle manufacturing system using a laminated manner according to an embodiment of the present invention and conceptually illustrates a configuration of a system for performing the above description in FIGS. 1 to 3.

Referring to FIG. 4, a system 400 according to the present invention includes a first nozzle device 410, a second nozzle device 420, and a controller 430. Of course, in the system according to the present invention, configurations for performing a laminated manner, for example, a base on which microneedle is formed, and a conveyor belt or a motor for moving chambers and the base, are omitted.

The first nozzle device 410 extrudes a first material on the base using a first nozzle.

Here, the first nozzle device 410 may extrude the first material on the base based on a predetermined extrusion sequence.

The second nozzle device 420 extrudes a second material, for example, a vaccine mixture on the base using a second nozzle.

Here, the second nozzle device 420 may extrude the second material on the base based on a predetermined extrusion sequence, and specifically, the second nozzle device may extrude the second material on the first material extruded on the base according to the extrusion sequence.

The controller 430 may be a configuration means for controlling the system according to the present invention, control the first nozzle device 410 and the second nozzle device 420 to perform extrusion of the first material and extrusion of the second material, and control movement of the base, the first nozzle device, and the second nozzle device.

Further, the controller 430 manufactures the microneedle using the first material and the second material extruded from the first nozzle device 410 and the second nozzle device 420, respectively, through a laminated manner, for example, 3D printing technology.

Here, the controller 430 may control a configuration means of the system including the first nozzle device 410 and the second nozzle device 420 to manufacture microneedle with a layered structure in which the first material and the second material are stacked.

In addition, the controller 430 may manufacture the microneedle through the laminated manner that reflects a mixing ratio of the first material and the second material and may manufacture the microneedle including more materials as well as two materials depending on a situation. Of course, when a microneedle including three or more materials is manufactured, three or more chambers are required and an extrusion sequence process is also required according thereto.

Although a description of the system of FIG. 4 is omitted, it is apparent to those skilled in the art that the system according to the present invention is capable of including all the above description in FIGS. 1 to 3.

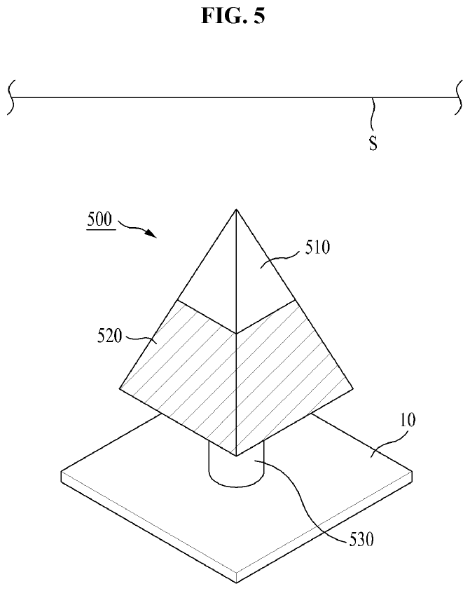

FIG. 5 is a perspective view illustrating a microneedle according to another embodiment of the present invention.

Referring to FIG. 5, a microneedle 500 according to another embodiment of the present invention includes an upper portion 510, a middle portion 520, and a lower portion 530.

The upper portion 510 is located at an upper end of the middle portion 520 to facilitate penetration into the skin “S”. The upper portion 510 may have a fore-end having a pointed tip shape based on a penetration direction into the skin “S”, which is formed in a pyramidal shape for example, a triangular, square, pentagonal, and hexagonal, or a conical shape to facilitate the penetration into the skin “S”. Here, it is characterized in that the upper portion 510 is made of a material having a stronger strength than those of the middle portion 520 and the lower portion 530 to facilitate the perforation of the skin “S”.

The upper portion 510 according to another embodiment of the present invention may allow the microneedle 500 to easily penetrate into the skin “S” and protect the middle portion 520 formed of a compound containing a drug component.

According to an embodiment, the upper portion 510 may be formed of a water-soluble material that penetrates and melts into the skin “S”. For example, the water-soluble material may be at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropylmethylcellulose (HPMC), ethyl cellulose (EC), hydroxypropyl cellulose (HPC), carboxymethylcellulose, cyclodextrin, and gentiobiose.

The middle portion 520 is capable of penetrating into the skin “S” through the upper portion 510, and is formed of the compound containing the drug component. The middle portion 520 is formed of the compound containing the drug component, and is solidified. Accordingly, when the middle portion 520 penetrates into the skin “S” by the upper portion 510, the solidified drug component may be melted and absorbed into the skin “S”.

The middle portion 520 of the microneedle 500 according to another embodiment of the present invention may be formed of the compound containing the drug component, that is, solidified, but may be a shape including a cavity, which is capable of including a drug in a liquid state depending on embodiments. It may be a shape including a cavity (cavity).

The middle portion 520 may have a truncated pyramidal shape, such as a triangular, square, pentagonal, and hexagonal, or a truncated cone shape from which the upper portion 510 is removed, and include a cavity area that is capable of containing a drug therein, and the drug may be solidified. Here, the cavity area may be preferably located at an upper end area above a center of the middle portion 520. However, a location, size, and shape of the cavity area are applied in various ways depending on a time when the drug is administered, a time of administration, and an amount to be administered. Furthermore, the size and location of the cavity may be adjusted depending on the amount of the drug, a evaporation rate and temperature, the shape of the middle portion 520 for the manufacturing the microneedle 500, a viscosity of the drug, a concentration of the drug, a solvent used, and a thickness covering a top of the cavity.

The middle portion 520 may be formed of a water-soluble material in the same manner as the upper portion 510 penetrating into the skin “S”. However, the middle portion 520 is formed of the compound containing the drug component, and thus it is preferable to use a material different from the upper portion 510 and the lower portion 530.

Here, the drug component of the middle portion 520 may be formed of a biocompatible material and an additive. For example, the biocompatible material may include at least one of carboxymethylcellulose (CMC), hyaluronic acid (HA), alginic acid, pectin, carrageenan, chondroitin sulfate, dextran sulfate, chitosan, polylysine, carboxymethyl chitin, fibrin, agarose, pullulan, polyanhydride, polyorthoester, polyetherester, polyesteramide, poly butyric acid, poly valeric acid, polyacrylate, ethylene-vinyl acetate polymer, acrylic substituted cellulose acetate, polyvinyl chloride, polyvinyl fluoride, polyvinyl imidazole, chlorosulfonate polyolefin, polyethylene oxide, polyvinylpyrrolidone (PVP), hydroxypropylmethylcellulose (HPMC), ethylcellulose (EC), hydroxypropylcellulose (HPC), carboxymethyl cellulose, cyclodextrin, maltose, lactose, trehalose, cellobiose, isomaltose, turanose, and lactulose, or at least one of a copolymer of monomers forming the polymer and cellouse.

In addition, the additives may include at least one of trealose, oligosaccharides, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropylmethylcellulose (HPMC), ethyl Cellulose (EC), hydroxypropyl cellulose (HPC), carboxymethyl cellulose, cyclodextrin, gentibiose, alkyltrimethylammonium bromide cetrimide, cetyltrimethylammonium bromide (CTAB), gentian violet, benzethonium chloride, docusate sodium salt, span-type surfactant polysorbate (Tween), sodium dodecyl sulfate (SDS), benzalkonium chloride, and glyceryl oleate.

In addition, the drug component of the middle portion 520 may be formed by mixing the biocompatible material and an active principle. The active principle includes a protein/peptide drug, but is not limited thereto, and includes at least one of a hormone, a hormone analog, an enzyme, an enzyme inhibitor, a signaling protein or a portion thereof, an antibody or a portion thereof, a single chain antibody, a binding protein or a binding domain thereof, an antigen, an adhesion protein, a structural protein, a regulatory protein, a toxin protein, cytokine, a transcriptional regulatory factor, a blood coagulation factor, and a vaccine. In detail, the protein/peptide drug may include at least one of insulin, insulinlike growth factor 1 (IGF-1), growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor TNF (TNF), atobisban, buserelin, cetrorelix, deslorelin, desmopressin, dynorphin A (1-13), elcatonin, eleidosin, eptifibatide, growth hormone releasing hormone-II (GHRHII), gonadorelin, goserelin, hisstrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormonereleasing hormone (LHRH), nafarelin, parathyroid hormone, pramlintide, T-20 (enfuvirtide), thymalfasin, and ziconotide.

In addition, as solvent of the drug component of the middle portion 520 may dissolve the biocompatible material. The solvent may include at least one of inorganic and organic solvents including DI water, methanol, ethanol, chloroform, dibutyl phthalate, dimethyl phthalate, ethyl lactate, glycerin, isopropyl alcohol, lactic acid, and propylene glycol.

The microneedle 500 according to another embodiment of the present invention may form the cavity of a specific area inside the middle portion 520 and include the liquid drug in the cavity to be injected into the skin “S”, and thus, it may be characterized in that the drug is administered in a dose. Accordingly, the present invention may enhance preservation of the drug, facilitate penetration into the skin, and allow the liquid drug to be administered.

The lower portion 530 supports the middle portion 520. The lower portion 530 has a prismatic shape, such as a triangular, square, pentagonal, and hexagonal, or a cylindrical shape, and supports the upper portion 510 and the middle portion 520.

The lower portion 530 may have a certain diameter and a certain height, which represent a degree of depth at which the microneedle 500 penetrates into the skin “S”. For example, the depth at which the upper portion 510 and the middle portion 520 including the drug penetrate into the skin “S” may be measured depending on the diameter and height of the lower portion 530 and the height of the lower portion 530 may be adjusted depending on the degree of depth at which the drug is to be penetrated, based on a type of drug, a state of the drug, the time the drug is administered, the time of administration, and the amount administered. In addition, the lower portion 530 may be adjusted in diameter depending on weights and sizes of the upper portion 510 and the middle portion 520, a degree to which the drug is capable of being supported, and a time for the lower portion 530 to melt inside the skin “S”.

It is characteristics in that the lower portion 530 is formed of a melting material that connects a base portion 10 and the microneedle 500 and the microneedle 500 is separated from the base portion 10. For example, the lower portion 530 may be formed of a water-soluble material to be rapidly dissolved, and thus the microneedle 500 formed on the base portion 10 may be quickly separated.

Here, the lower portion 530 may be formed of a water-soluble material in the same manner as the upper portion 510 and the middle portion 520 penetrated into the skin “S”. However, the lower portion 530 may be formed of a material that melts faster than those of the upper portion 510 and the middle portion 520 among water-soluble materials. The upper portion 510 is for easier skin perforation, the middle portion 520 is for more efficient administration of the drug, and the lower portion 530 is for quick separation of the microneedle 500 formed on the base portion 10 and the depth degree of the microneedle 500 into the skin “S”, and thus it is characteristics in that the microneedle 500 according to another embodiment of the present invention includes the upper portion 510, the middle portion 520, and the lower portion 530, which have three or more layers formed of different materials from one another, respectively.

The lower portion 530 according to another embodiment of the present invention may serve to support the upper portion 510 and the middle portion 520 in the microneedle 500 and indicate the degree of penetration depth into the skin. As shown in FIG. 5, it is characteristics in that the lower portion 530 has a prismatic or cylindrical shape and occupies a size and volume smaller than those of the upper portion 510 and the middle portion 520. Therefore, the lower portion 530 minimizes an area, volume, and weight of the microneedle 500 and shows an effect of supporting to administer the dose due to the appropriate size, height, and diameter of the lower portion 530 depending on the degree of penetration depth of the microneedle 500 into the skin “S”.

As shown in FIG. 5, the microneedle 500 may be formed on the base portion 10. The base portion 10 is not provided with the drug and is detachable after the microneedle 500 of the upper portion 510, the middle portion 520, and the lower portion 530 penetrate into the skin “S”. For example, the base portion 10 is provided in a form of a kind of patch to be capable of being in close contact with the skin “S”.

The base portion 10 may be formed of a water-insoluble material that does not melt, unlike the microneedle 500 that penetrates into the skin “S”. Accordingly, the base portion 10 may not interfere with penetrating force of the microneedle 500 to guide supply of the drug in a dose included in the middle portion 520.

For example, the base portion 10 may be formed with at least one from a group consisting of polyethylene (PE), polypropylene (PP), polytetrafluoroethylene (PTFE), polymethyl methacrylate (PMMA), ethylene vinyl acetate (EVA), polycapro Lactone (PCL), polyurethane (PU), polyethylene terephthalate (PET), polyethylene glycol (PEG), polyvinyl alcohol (PVA), polylactide (PLA), polylactide-glycolide copolymer (PLGA), and polyglycolide (PGA).

As shown in FIG. 5, the microneedle 500 according to another embodiment of the present invention is formed to have three or more layers structure of a tree shape, which includes the middle portion 520 formed of the compound containing the drug component, the upper portion 510 disposed at the upper end of the middle portion 520 to facilitate penetration into the skin “S”, and the lower portion 530 supporting the upper portion 510 and the middle portion 520 and facilitating the separation from the base portion 10, thereby being capable of strengthening the preservation of the drug, facilitating the penetration into the skin, and administering the drug in dose.

In addition, the microneedle 500 according to another embodiment of the present invention may have the tree-shaped three or more layer structure to minimize penetration resistance due to skin elasticity when attached to the skin, thereby increasing a penetration rate of the structure (60% above) and an absorption rate of the active principle into the skin. In addition, the tree-shaped microneedle 500 applies the three or more layers structure to maximize mechanical strength, thereby facilitating skin penetration.

In addition, it is characteristics in that the upper portion 510 and the middle portion 520 of the conical or pyramidal shape and the lower portion 530 of the prismatic or cylindrical shape, which forms the microneedle 500 according to another embodiment of the present invention, are manufactured by the 3D printing technology. The present invention may use the 3D printing manner to have the very short attachment time, the high precision, and the low price while the number density of the microneedle in the micro-patch may be increased and the aspect ratio may be improved, compared to the conventional manner.

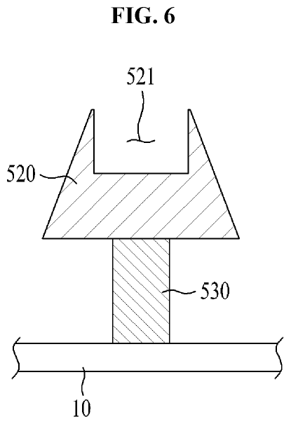

FIG. 6 is a cross-sectional view of a microneedle including a cavity according to another embodiment of the present invention.

The microneedle 500 according to another embodiment of the present invention may basically include the middle portion 520 formed of the compound containing the drug component, that is, the solidified material. However, the microneedle 500 may include the middle portion where a cavity 521 is formed to contain a liquid drug according to an embodiment to be applied. Accordingly, hereinafter, a description will be given of the middle portion 520 including the cavity 521 according to an embodiment.

Referring to FIG. 6, the microneedle 500 according to another embodiment of the present invention may include the middle portion 520 with the cavity 521. The cavity 521 may be formed in a shape of a groove in the middle portion 520 and formed in a shape and size for containing a drug.

Here, a surface of the cavity in contact with the drug may be coated with a waterproof material. When the microneedle 500 according to another embodiment of the present invention includes the cavity 521, the drug in a liquid state may be included. Accordingly, it is characteristics in that the surface of the cavity is coated with the waterproof material to block absorption of the drug in the middle portion 520.

For example, the cavity surface may be coated with a waterproofing agent including a mineral-based material or a lipid-based material. Here, the waterproofing agent may include at least one or more of beeswax, oleic acid, soy fatty acid, castor oil, phosphatidylcholine, d-α-tocopherol/vitamin E, corn oil mono-ditridiglyceride, cottonseed oil, olive oil, peanut oil, peppermint oil, safflower seed oil, sesame oil, soybean oil, hydrogenated vegetable oil, hydrogenated soybean oil, caprylic/capric triglycerides derived from coconut oil or palm see oil, and phosphatidylcholine (Phosphatidylcholine), or may be formed from a mixture thereof.

Depending on embodiments, the cavity surface may be coated with different waterproofing agents depending on a type and state of the drug injected into the cavity 521 and the cavity 521 may be formed in the middle portion 520 in different sizes, heights, shapes depending on type of drug, state of drug, timing of drug administration, administration time, and amount administered.

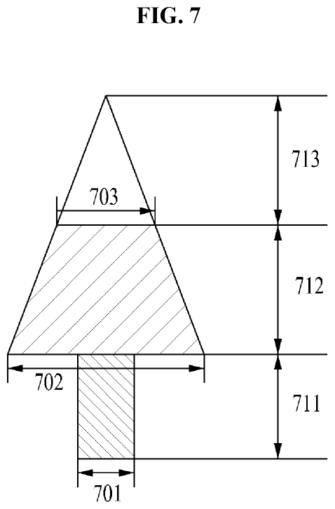

FIG. 7 is a cross-sectional view of a microneedle of three or more layers structure according to another embodiment of the present invention.

The microneedle according to another embodiment of the present invention, which is a microstructure composed of a three or more layers structure, includes the upper portion and the middle portion, which have the pyramidal or conical shape, and the lower portion of the prismatic or cylindrical shape.

As shown in FIG. 7, it is characteristics in that a bottom diameter 702 of the middle portion is greater than a bottom diameter 703 of the upper portion or a bottom diameter 701 of the lower portion, and the bottom diameter 703 of the upper portion is the bottom diameter 701 of the lower portion. The size may be determined in the order of the bottom diameter 702 of the middle portion, the bottom diameter 703 of the upper portion, and the bottom diameter 701 of the lower portion.

In addition, a height 712 of the middle portion may be higher than a height 713 of the upper portion, and a sum height of the height 712 of the middle portion and the height 713 of the upper portion may be higher or lower than a height 711 of the lower portion. That is, in the microneedle 500 according to another embodiment of the present invention, the height 712 of the middle portion may be the highest, and the height 713 of the upper portion and the height 711 of the lower portion may be the same as each other, and may be different from each other depending on an embodiment to which the microneedle 500 according to another embodiment of the present invention is applied. Meanwhile, the height 711 of the lower portion, the height 712 of the middle portion, and the height 713 of the upper portion of the microneedle 500 according to another embodiment of the present invention are not limited thereto shown in FIG. 7 and may have various heights depending on embodiments.

The middle portion of the microneedle according to another embodiment of the present invention may be formed with the cavity containing the drug, thereby being formed with the widest volume, the largest bottom diameter 702, and the highest height 712. The bottom diameter 703 of the upper portion, which has the pyramidal or conical shape for penetrating the skin “S”, may be the same as a diameter of an upper surface (or fore-end) of the middle portion and may be determined by a cross-sectional area of the fore-end of the truncated pyramid or truncated cone forming the middle portion. In addition, the height 713 of the upper portion may be determined depending on the shape of the truncated pyramid or truncated cone of the middle portion.

The lower portion of the microneedle according to another embodiment of the present invention, which serves to support the upper portion and the middle portion in the microneedle, may represent the depth of penetration into the skin. Accordingly, the lower portion 530 has the smaller volume and lower diameter 701 than those of the upper portion and the middle portion. However, the height 711 of the lower portion may be determined depending on the depth of penetration into the skin.

It is characteristics in that the lower portion of prismatic or cylindrical shape have the bottom diameter 701 smaller than the bottom diameter 703 of the upper portion and the bottom diameter 702 of the middle portion, and the volume smaller than those of the upper portion and the middle portion. It is characteristics in that the lower portion represents the degree of depth into the skin “S” and is for supporting the upper portion and the middle portion, thereby minimizing the area, volume, and weight of the microneedle according to another embodiment of the present invention. Accordingly, the lower portion has an effect of ensuring that the dose of the drug is capable of being administered, due to the shape with appropriate size, height, and diameter depending on the depth of the microneedle penetrating into the skin “S”.

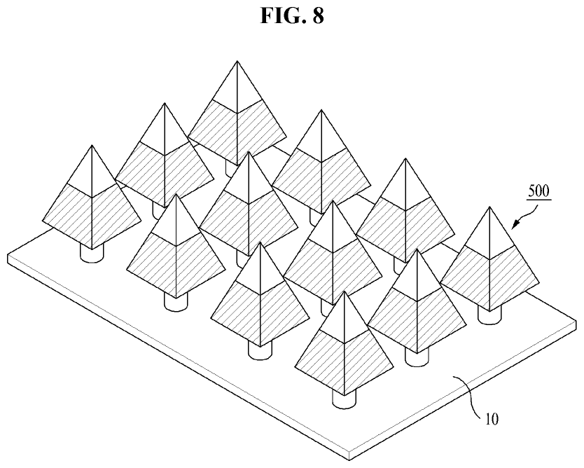

FIG. 8 shows a perspective view of a microneedle patch manufactured according to another embodiment of the present invention.

As shown in FIG. 8, a plurality of microneedles 500 manufactured by the above description may be formed on the base 10 to manufacture a microneedle patch and be easily applied to the medical field. That is, the present invention may manufacture the microneedle 500 having the three or more layers structure by the laminated manner using the 3D printing to secure high competitiveness in the medical market field.

The system or device described above may be implemented using a hardware component, a software component, or a combination of the hardware component and software component. For example, the system, device and elements described in embodiments may be configured using one or more general-purpose or special purpose computers, such as, for example, a processor, a controller and an arithmetic logic unit (ALU), a digital signal processor, a microcomputer, a field programmable array (FPA), a programmable logic unit (PUL), a microprocessor, or any other device capable of responding to and executing an instruction. The processing device may run an operating system (OS) and one or more software applications that run on the OS. In addition, the processing device also may access, store, manipulate, process, and create data in response to execution of the software. For purpose of simplicity, the description of a processing device is used as singular; however, one skilled in the art will appreciate that a processing device is capable of being including multiple processing elements and multiple types of processing elements. For example, a processing device may include multiple processors or a processor and a controller. In addition, different processing configurations are possible, such as parallel processor.

The software may include a computer program, a piece of code, an instruction, or some combination thereof, and configure the processing device to operate as desired or independently or collectively instruct the processing device. Software and/or data may be embodied permanently or temporarily in any type of machine, component, physical or virtual equipment, computer storage medium or device, or in a propagated signal wave, which is for being interpreted by the processing device or providing instructions or data to the processing device. The software also may be distributed over network coupled computer systems to be stored or executed in a distributed fashion. The software and data may be stored by one or more computer readable recording media.

The methods according to embodiments may be implemented in the form of program instructions that are capable of being executed through various computer means to be recorded in computer-readable media. The computer-readable media may include, alone or in combination with program instructions, data files, data structures, and the like. The program instructions recorded on the media may be specially designed and configured for the embodiment, or may be known to and usable by those skilled in computer software arts. Examples of the computer-readable media include magnetic media such as hard disks, floppy disks, and magnetic tape, optical media such as CD ROM disks and DVD, magneto-optical media such as floptical disks, and hardware devices that are specially configured to store and execute program instructions such as read-only memory (ROM), random access memory (RAM), flash memory, and the like. Examples of program instructions include higher level code that is capable of being executed by the computer using an interpreter as well as machine code such as produced by a compiler. The described hardware devices may be to act as one or more software modules in order to perform the operations of the above-described embodiment or vice versa.

While the above-described embodiments have been described with reference to exemplary embodiments and drawings, those skilled in the art can make various changes and modifications from the above description. For example, suitable results may be achieved although the described techniques are performed in a different order, and/or the components in the described system, architecture, device, or circuit are coupled or combined in a different manner, or are replaced or supplemented by other components or their equivalents.

Therefore, other implements, other embodiments, and equivalents to claims are within the scope of the following claims.

What is claimed is:

1. A method of manufacturing a microneedle, the method comprising:
extruding a first material using a first nozzle and extruding a second material using a second nozzle; and
manufacturing the microneedle through a laminated manner using the extruded first material and second material.

2. The method of claim 1, wherein the manufacturing of the microneedle includes manufacturing the microneedle through a 3D printing manner using the first material and second material.

3. The method of claim 1, wherein the extruding includes extruding the first material by a predetermined first extrusion sequence and extruding the second material by a predetermined second extrusion sequence.

4. The method of claim 1, wherein the manufacturing of the microneedle includes manufacturing the microneedle through a laminated manner reflecting a mixing ratio of the first material and the second material.

5. A method of manufacturing a microneedle, the method comprising:
extruding a plurality of materials using at least two or more nozzles; and
manufacturing the microneedle through a laminated manner using the extruded plurality of materials.

6. A microneedle patch comprising a microneedle manufactured by the method of claim 1.

7. A microneedle manufacturing system comprising:
a first nozzle device for extruding a first material using a first nozzle;
a second nozzle device for extruding a second material using a second nozzle; and
a controller configured to manufacture the microneedle through a laminated manner using the extruded first material and second material.

8. The microneedle manufacturing system of claim 7, wherein the controller manufactures the microneedle through a 3D printing manner using the first material and second material.

9. The microneedle manufacturing system of claim 7, wherein the first nozzle device extrudes the first material by a predetermined first extrusion sequence, and
wherein the second nozzle device extrudes the second material by a predetermined second extrusion sequence.

10. The microneedle manufacturing system of claim 7, wherein the controller manufactures the microneedle through a laminated manner reflecting a mixing ratio of the first material and the second material.

11. A microneedle of three or more layer structure, the microneedle comprising:
a middle portion configured to penetrate into a skin and be formed of a compound containing a drug component;
a lower portion configured to support the middle portion; and
an upper portion disposed on an upper end of the middle portion to facilitate penetration.

12. The microneedle of claim 11, wherein the upper portion and middle portion have a pyramidal or conical shape, and the lower portion has a prismatic or cylindrical shape.

13. The microneedle of claim 12, the middle portion has a bottom diameter, which is greater than a bottom diameter of the upper portion or a bottom diameter of the lower portion, and
wherein the bottom diameter of the upper portion is larger than the bottom diameter of the lower portion.

14. The microneedle of claim 11, wherein the upper portion has a height and a bottom diameter, which are determined by a cross-sectional area of a fore-end of a truncated pyramid or truncated cone of the middle portion.

15. The microneedle of claim 12, wherein the lower portion is located in the lowermost layer of the three or more layers structure to be in a shape coupled to a bottom diameter of a pyramid or cone of the middle portion, and is formed with a diameter capable of supporting the middle portion formed of a compound containing a drug component in company with the upper portion.

16. The microneedle of claim 11, wherein the lower portion is formed of a melting material connecting a base portion to the microneedle to separate the microneedle from the base portion.

17. The microneedle of claim 11, wherein the middle portion is formed of a compound containing a drug component and is solidified.

18. The microneedle of claim 11, wherein the upper portion, the middle portion, and the lower portion are formed of different materials.

19. The microneedle of claim 11, wherein the microneedle is manufactured through a 3D printing manner.

20. A microneedle patch comprising a microneedle manufactured by the method of claim 2.
